(19) United States
(12) Patent Application Publication
(10) Pub. No.: US 2005/0148949 A1
(43) Pub. Date: Jul. 7, 2005

(54) Novel instrument for the transplantation of delicate micro-transplants

(76) Inventor: Gabrielle Thumann, Cologne (DE)

Correspondence Address: DLA PIPER RUDNICK GRAY CARY US, LLP, 4365 EXECUTIVE DRIVE, SUITE 1100, SAN DIEGO, CA, 92121-2133, US

(21) Appl. No.: 10/903,114

(22) Filed: Jul. 30, 2004

Related U.S. Application Data
(60) Provisional application No. 60/491,174, filed on Jul. 31, 2003.

(51) Int. Cl. A61M 29/00

(52) U.S. Cl. 604/264

(57) ABSTRACT

An instrument and method is disclosed for delivering an explant at the site of implantation. The instrument affords the explant protection from damage and loss during transit to the implantation site, while allowing for the proper positioning and orientation of the explant.

### RELATED APPLICATION DATA

This application claims the benefit of U.S. Provisional Application No. 60/491,174, filed Jul. 31, 2003, titled, A NOVEL INSTRUMENT FOR THE TRANSPLANTATION OF DELICATE MICRO-TRANSPLANTS, the contents of which are incorporated by reference in their entirety.

### FIELD OF INVENTION

The present invention relates to design and manufacture of an instrument suitable for transplanting and positioning micro-transplants of delicate and/or viable tissues in a subject. The instrument is designed to collect, hold in place and deliver a micro-explant through tissues to the site of transplant without subjecting the micro-explant to undue damage.

### BACKGROUND OF THE INVENTION

Transplantation of tissues to normally unexposed sites requires that the explant be transported to the site through other tissues. Two problems are encountered during the transport of the explant, namely the explant can be lost during transport to become localized at the incorrect site and the explant can become damaged during the transfer. One way to obviate these problems would be to expose the site by surgical intervention; however, in many cases exposing the site of implantation is not possible because such a procedure would cause trauma and irreparable damage to other tissue and structures.

As an example, it has been hypothesized that transplantation of a retinal pigment epithelial cell sheet to the macular region of the subretinal space would be necessary to treat macular degeneration. However, to expose the site where the explant should be positioned without causing damage to the explant, would requires exposure of the subretinal space. Exposure of the subretinal space would cause irreparable damage to many ocular structures including the retina with probable loss of vision.

Transplantation of tissues or other substances to unexposed areas of the brain would likewise require surgical procedure that would produce large defects, with the probability of irreparable damage to functional structure and thus loss of functionality.

The use of the various instruments now being used to transport explants, such as forceps of various configurations have the disadvantage of damaging the explant, probable loss of the explant during the transit to the site of implantation and the possible creation of large defects to surrounding structures.

Steinhorst U H et al. (Subretinal transplantation of confluent pigment epithelium: novel devise for non-traumatic tissue handling. Klin Monatsbl Augenheilkd 214: 103-106; German Pat. Reg. No. 29819018.4) have devised an instrument to transport explants to the subretinal space. This instrument is a perforated spoon into which the tissue can be placed and held in place by vacuum. However, the instrument described by Steinhorst et al suffers from two limitations: first, the instrument does not protect the explant but leaves it exposed to the stress and sheer of the medium through which the instrument must traverse, the sclerotomy and the vitreous, thus exposing it to damage and possible loss during the transit through the sclera and the vitreous. Second, the shape of the area that carries the explant is concave, which would not be suitable for a rigid explant and could lead to damage of a very delicate explant, when the vacuum applied to hold the explant in place would necessarily distort the explant to fit the concave surface.

### SUMMARY OF THE INVENTION

The present invention overcomes these and other problems in the art by providing an instrument that protects the explant from loss and damage during transport to the implantation site and by providing a flat surface for the explant. The instrument should also provide the explant with an environment appropriate to any possible explant.

Accordingly, in one embodiment, the invention provides methods for the implantation to the subretinal space of a biological membrane with retinal pigmented cells attached in order to replace degenerated retinal pigment epithelial cells.

In yet another embodiment, the invention provides methods for ameliorating and/or restoring the loss of visual acuity in patients with degenerated and/or damaged retinal pigment epithelium by transplanting “sheets” of retinal or iris pigment epithelium to the macular area of the retina.

In yet another embodiment, the invention provides methods for the transport of explants to the corpus callosum of the brain guided by a stereotactic instrument.

### BRIEF DESCRIPTION OF THE FIGURES

The drawings accompanying and forming part of this specification are included to depict certain aspects of the invention. A clearer conception of the invention, and of the components and operation of systems provided with the invention, will become more readily apparent by referring to the exemplary, and therefore nonlimiting, embodiments illustrated in the drawings, wherein identical reference numerals designate the same components. The invention may be better understood by reference to one or more of these drawings in combination with the description presented herein. It should be noted that the features illustrated in the drawings are not necessarily drawn to scale

FIG. 1 shows one embodiment of an instrument for the transplantation of delicate micro-transplants.

FIG. 2 shows details of the carrier platform end of the instrument.

FIG. 3 shows a thin transparent membrane upon the carrier platform.

FIG. 4 shows a stained membrane on the carrier platform in the vitreous after retraction of the protective cannula.

FIG. 5 shows the stained membrane positioned in the subretinal space.

### DETAILED DESCRIPTION OF THE INVENTION

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, use of the “a” or “an” are employed to describe elements and components of the invention. This is done merely for convenience and to give a general sense of the invention. This description should be read to include one or at least one and the singular also includes the plural unless it is obvious that it is meant otherwise.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Embodiments of the instrument or device and methods for fabricating and using the device are described in detail herein. In the following description, numerous specific details are provided, such as the identification of various system components, to provide an understanding of embodiments of the invention. One skilled in the art will recognize, however, that embodiments of the invention can be practiced without one or more of the specific details, or with other methods, components, materials, etc. In still other instances, well-known structures, materials, or operations are not shown or described in detail to avoid obscuring aspects of various embodiments of the invention.

Reference throughout this specification to “one embodiment” or “an embodiment” means that a particular feature, structure, or characteristic described in connection with the embodiment is included in at least one embodiment of the present invention. Thus, the appearance of the phrases “in one embodiment” or “in an embodiment” in various places throughout this specification are not necessarily all referring to the same embodiment. Furthermore, the particular features, structures, or characteristics may be combined in any suitable manner in one or more embodiments.

As an overview, one embodiment of the invention provides methods for the construction of an instrument 100 for the transport of an explant to a subretinal space. The instrument consists of a stainless cannula 120 of 1.3 mm in diameter and 150 mm in length, with one end shaped into a closed, perforated platform (the carrier platform 135) upon which a cell sheet, a tissue or any type of explant (e.g., a sponge containing a growth factor, a slow-release carrier that would release an inhibitor of vascularization) can be placed. Five mm from the end opposite of the carrier platform is welded a threaded cap, 2.5 mm in diameter to which a syringe is attached via a plastic tubing 125. This first cannula 120 is fitted into a second stainless steel cannula 115 (2.0 mm in diameter) that is fitted with a handle 105 to allow the sliding of this cannula 120 forward and backward. The cannulas 115, 120 are fitted into an instrument handle 105 that has a slit on its side through which the handle 110 of the second cannula 115 is inserted.

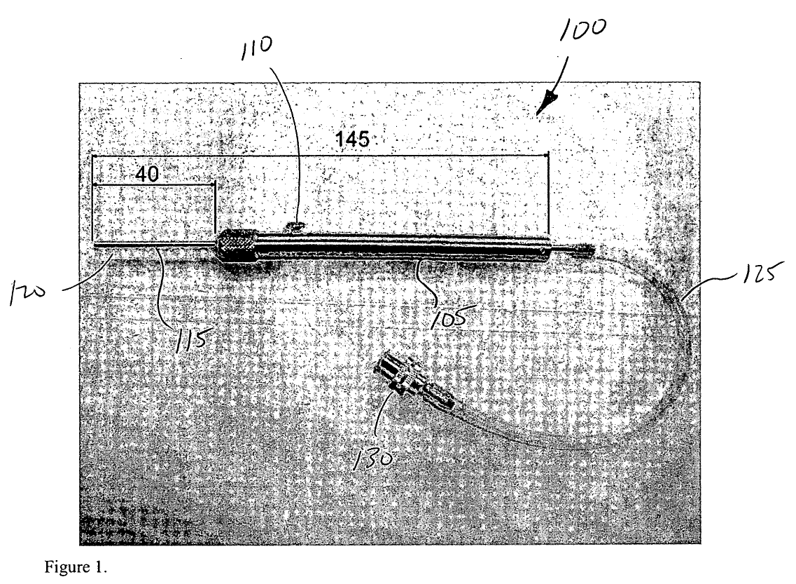

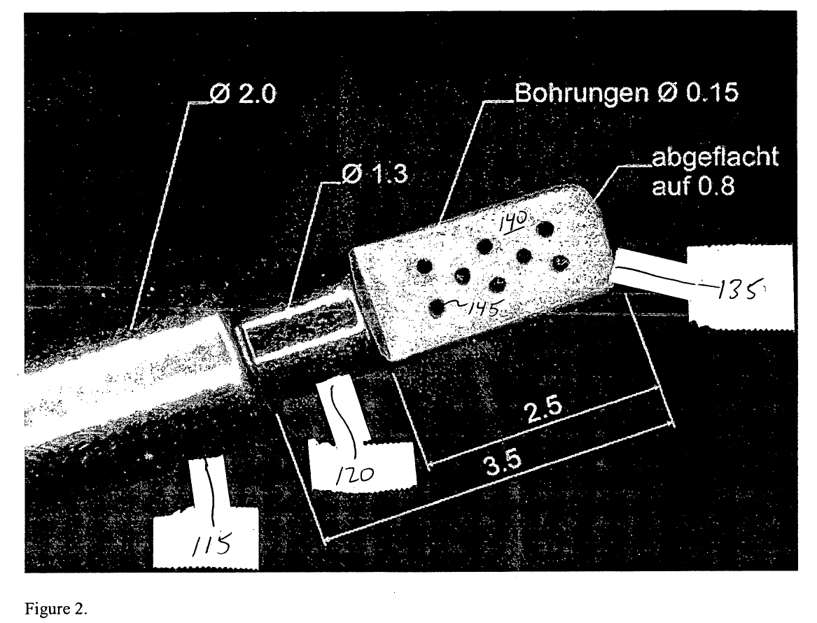

FIG. 1 is a photograph showing one embodiment of the instrument, as it would appear with the cannula 115 extended to protect an explant on the carrier platform 135. In this specific configuration, the instrument 100 comprises an outer tube or handle 105 having a slit trough which is fitted a handle 110 that is used to slide an inner protective cannula 115 over a holding cannula 120. In the embodiment shown, the total length of the tube and cannula is 145 mm and the 2 mm diameter protective cannula 115 extends 40 mm from the end of the holding cannula 105. Note the handle 110, which is used to slide the 2.0 mm diameter protective cannula 120. A plastic tubing 125 fitted with a Lauer lock 130 is attached to the far end of the 2 mm protective cannula 110. The lock may be attached to a syringe. Retracting the protective cannula 115 reveals the holding cannula 120 with a carrier platform 135 (see FIG. 2).

FIG. 2 illustrates one embodiment of the holding cannula 120 and carrier platform 135. The carrier platform 135 has a perforated floor 140 upon which the explant is positioned and held in place by negative pressure exerted by pulling on the syringe (not shown) attached to the lock 130. In this configuration, the total length of the carrier platform 135 is 2.5 mm in length and 0.8 mm in width. The perforated floor 140 has 8 round perforations 140, each of 0.15 mm in diameter. The 2.0 mm protective cannula 115 can be retracted to uncover the holding cannula 120, 1.5 mm in diameter, from which the carrier platform 135 is shaped. The carrier platform may be made from the flattened closed end of a cannula.

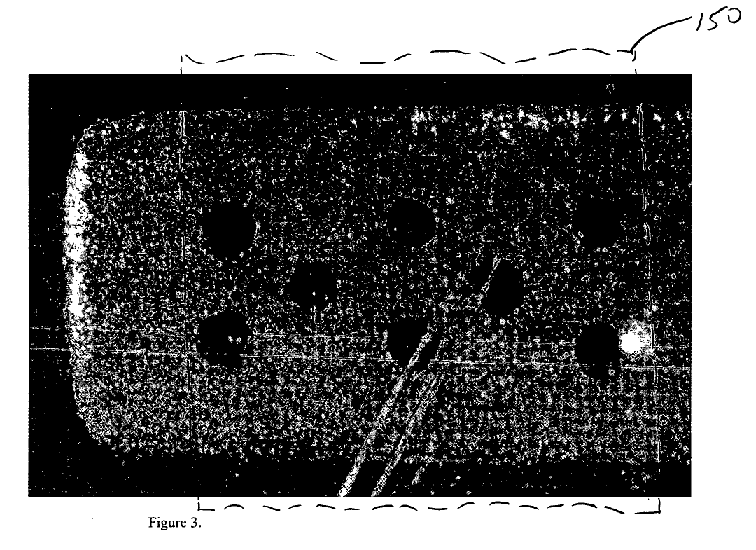

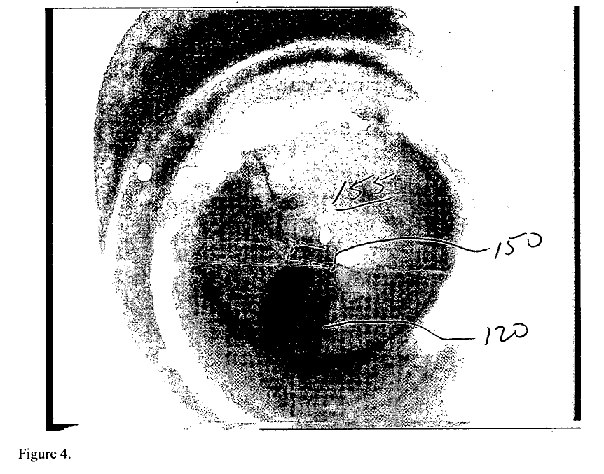

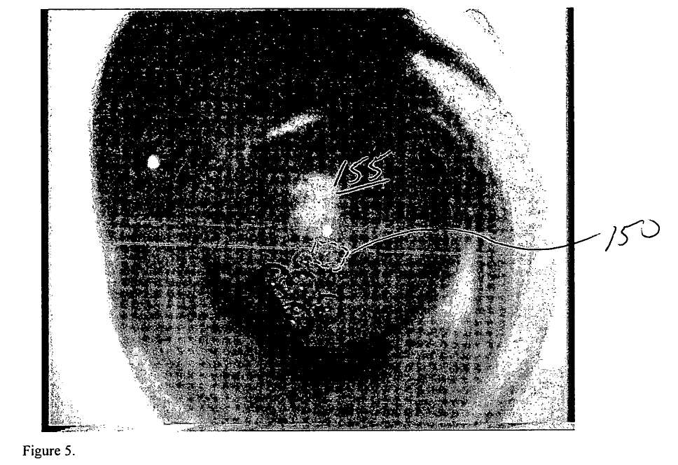

FIG. 3 illustrates the carrier platform 135 upon which rests a thin transparent membrane 150, which can be covered and thus protected by sliding forward the 2 mm diameter cannula 115. The injector instrument 100, with the protective cannula 115 pulled forward protecting the membrane 150, can be introduced into the vitreous through a sclerotomy without damaging or dislodging the membrane 150. In FIG. 4, a stained membrane 150 on the carrier platform is shown in the vitreous 155 after retraction of the protective cannula 115. FIG. 5 shows the stained membrane 150 positioned in the subretinal space.

In one embodiment, the manufacture of the instrument 100 is stainless steel, however in other embodiment, it can be manufactured of any non-toxic material compatible with the material to be explanted, such as plastic or glass. In the present invention negative pressure and release of the explant is affected by the use of a syringe, however a pump can be attached for accurate measurement of pressure. In the present invention, the instrument is straight, however it can be manufacture angled with a flexible protective cannula.

In another embodiment, the invention provides for methods to transfer an explant to an implantation site. To transplant the tissue, the inner cannula is filled with fluid, e.g., balanced salt solution, culture medium or other appropriate fluid; the explant is placed on the carrier platform 135 either with the aid of forceps or by placing the carrier platform underneath the tissue suspended in a fluid phase. Once the explant is seated on the carrier platform, it is secured in place by exerting a slight suction with the syringe. The carrier platform with the tissue is then covered by sliding the second cannula 115 over it, thus protecting the tissue from damage or dislodgement while it is introduced into a tissue or organ to reach the site of transplantation. FIG. 4 illustrates the instrument with the second cannula completely advanced covering the entire carrier platform. As the carrier platform holding the tissue is recessed, the walls of the second cannula do not come in contact with the tissue, and since the system is open the tissue is always bathed in a liquid appropriate to the tissue to be transplanted. FIG. 3 shows the carrier platform with a transparent unstained tissue foil in place.

The transplantation procedure has been established in enucleated porcine eyes. The porcine eye is placed under an operating microscope, a vitrectomy contact lens is placed on the cornea with 2% methylcellulose as contact medium to visualize the retina, and a sclerotomy is performed at the 11 o'clock position. A sharp 36-gauge needle is introduced into the eye to perform a retinotomy, and a retinal bleb is created by injecting a small amount of balanced salt solution through the retinotomy. The needle is then removed, and the sclerotomy is enlarged slightly to allow for the introduction of the injector instrument 100 (outside diameter 2.0 mm). The injector is introduced through the sclerotomy while the cannula 115 is still covering the carrier platform 135 with the explant secured in place. The cannula 115 covering the platform is then pulled back, the carrier platform 135 is advanced into the subretinal space, and the explant released by exerting a small amount of pressure with the syringe. Using the carrier platform 115, the explant is then manipulated into place.

In the vitreous cavity, the cannula 115 is pulled back, allowing for visual control of the sheet (FIG. 4). In addition, the diameter of the instrument is reduced to the diameter of the inner cannula 120, 1.3 mm, and can now be introduced through a small retinotomy into the subretinal space (FIG. 5). Using the end of the injector the explant can be manipulated in its proper place.

Although the foregoing has been described in some details by way of illustration and example, for the purpose of clarity and understanding, it is obvious that certain changes and modifications may be practiced within the scope of the appended claims.

While the invention is described and illustrated here in the context of a limited number of embodiments, the invention may be embodied in many forms without departing from the spirit of the essential characteristics of the invention. The illustrated and described embodiments, including what is described in the abstract of the disclosure, are therefore to be considered in all respects as illustrative and not restrictive. The scope of the invention is indicated by the appended claims rather than by the foregoing description, and all changes that come within the meaning and range of equivalency of the claims are intended to be embraced therein.

1. A device for the safely transporting an explant to the site of implantation, comprising:
an instrument that has a carrier platform;
means to hold the explant in place on the carrier platform;
means to release the explant from the carrier platform; and
means to protect the explant during transit to the site of implantation.

2. The device of claim 1 wherein the instrument is straight.

3. The device of claim 1 wherein the instrument is angled.

4. The device of claim 1 wherein the carrier platform is the flattened closed end of a holding cannula.

5. The device of claim 1 wherein the carrier platform is perforated.

6. The device of claim 1 wherein the holding cannula with the carrier platform is fitted with a syringe attached via plastic tubing.

7. The device of claim 1 wherein the carrier platform is fitted with a vacuum pump.

8. The device of claim 1 wherein the carrier platform is protected by sliding over it a second larger cannula.

9. The device of claim 1 wherein said carrier platform comprises a stainless steel cannula.

10. The device of claim 1 wherein said carrier platform comprises a rigid plastic cannula.

11. The device of claim 1 wherein said carrier platform comprises a flexible plastic cannula.

12. The device of claim 1 wherein said carrier platform comprises a glass cannula.

13. The device of claim 1 wherein said means of protecting the explant comprises a second larger stainless steel cannula.

14. The device of claim 1 wherein said means of protecting the explant comprises a second larger rigid plastic cannula.

15. The device of claim 1 wherein said means of protecting the explant comprises a second larger flexible plastic cannula.

16. The device of claim 1 wherein said means of protecting the explant comprises a second larger glass cannula.

17. The device of claim 1 wherein the explant is a membrane with viable cells attached.

18. The device of claim 1 wherein the explant is a sponge containing a biological substance.

19. The device of claim 1 wherein the explant is a substance capable of releasing slowly a biological substance.

20. The device of claim 1 wherein the explant is a collagenous material with cells embedded in it.

21. A method for the safely transporting an explant to the site of implantation, comprising:
providing an instrument that has a carrier platform;
holding the explant in place on the carrier platform;
protecting the explant during transit to the site of implantation, and
releasing the explant from the carrier platform at the site of implantation.

22. The method of claim 21 wherein the carrier platform is the flattened closed end of a holding cannula.

23. The method of claim 21 wherein holding the explant in place on the carrier platform includes a perforated carrier platform connected to a vacuum source.

24. The method of claim 23 wherein the vacuum source is a syringe.

25. The method of claim 23 wherein the instrument further includes a larger protective cannula and protecting the explant comprises sliding the protective cannula over the holding cannula covering carrier platform.

26. The method of claim 25 wherein releasing the explant from the carrier platform includes sliding the protective cannula uncovering the carrier platform and releasing the vacuum.

27. The method of claim 26 further comprising applying pressure to the carrier platform.

28. An instrument for safely transporting an explant to the site of implantation, comprising:
a holding cannula with a perforated carrier platform;
a vacuum source fluidly connected to the holding cannula and carrier platform; and
a protective cannula sized to slide over the holding cannula and carrier platform

29. The instrument of claim 28 further comprising:
an outer tube having a slit, the outer tube being sized to fit the protective cannula; and
a handle, the handle being attached to the protective cannula through the slit.

30. The instrument of claim 28 wherein the carrier platform is a flattened closed end of the holding cannula.

31. The device of claim 28 wherein the vacuum source is a syringe.

32. The method of claim 28 wherein the holding cannula material is selected from the group consisting of stainless steel, rigid plastic, flexible plastic and glass.

33. The method of claim 28 wherein the protective cannula material is selected from the group consisting of stainless steel, rigid plastic, flexible plastic and glass.
